(19) United States
(12) Patent Application Publication
(10) Pub. No.: US 2024/0018550 A1
(43) Pub. Date: Jan. 18, 2024

(54) ADENINE BASE EDITOR HAVING INCREASED THYMINE-CYTOSINE SEQUENCE-SPECIFIC CYTOSINE EDITING ACTIVITY, AND USE THEREOF

(71) Applicants: IUCF-HYU (INDUSTRY-UNIVERSITY COOPERATION FOUNDATION HANYANG UNIVERSITY), Seoul (KR); Korea University Research and Business Foundation, Seoul (KR)

(72) Inventors: Sangsu BAE, Seoul (KR); You Kyeong JEONG, Boston, MA (US); Seokhoon LEE, Seoul (KR); Jae-Sung WOO, Seoul (KR)

(73) Assignees: IUCF-HYU (INDUSTRY-UNIVERSITY COOPERATION FOUNDATION HANYANG UNIVERSITY), Seoul (KR); Korea University Research and Business Foundation, Seoul (KR)

(21) Appl. No.: 18/039,630

(22) PCT Filed: Dec. 1, 2021
(86) PCT No.: PCT/KR2021/017954

§ 371 Date: May 31, 2023

(30) Foreign Application Priority Data
Dec. 1, 2020 (KR) 10-2020-0166045
May 12, 2021 (KR) 10-2021-0061103

(51) Int. Cl. C12N 15/90; C12N 15/11; C12N 9/22; C12N 9/24; C12N 9/78

(52) U.S. Cl. CPC C12N 15/90; C12N 15/11; C12N 9/22; C12N 9/2497; C12Y 302/02027; C12N 9/78; C12Y 305/04004; C12N 2310/20

(57) ABSTRACT

Proposed are an adenine base editor having increased thymine-cytosine sequence-specific cytosine base editing activity, a cytosine base editing method, and a cytosine base editing kit. The editor, produced by introducing a P48R mutation into an adenosine deaminase, has an operating range that is more sophisticated than conventional cytosine base editors, allows cytosine base editing only when cytosine is positioned right behind thymine, thereby enabling elaborate editing even when there is a plurality of cytosines within the range, and enables cytosine to be substituted with thymine or guanine in the presence or absence of UGI, respectively. Therefore, the cytosine base editing composition can be effectively used in the fields of gene therapy or new crop development which requires precise editing of only cytosine in all living organisms, including humans, plants, and bacteria.

### TECHNICAL FIELD

The present disclosure relates to an adenine base editor having increased thymine-cytosine sequence-specific cytosine base editing activity, to a composition for edition of a thymine-cytosine sequence-specific cytosine base, the composition containing the base editor and a guide RNA, to a cytosine base editing method, and to a cytosine base editing kit.

### BACKGROUND ART

Adenine base editors (ABEs) are effective gene editing tools capable of converting A/T pairs to G/C pairs without causing DNA double-strand breaks (DSBs) or requiring a donor DNA template. Such technology is used not only at the cell level but also when bases are edited in plants or animals. In addition, verification and development are being actively conducted so as to be used for gene therapy. An earlier practical version of ABE (ABE7.10) is composed of three fused elements, which is specifically composed of a pair of partially inactive Cas nuclease (Cas nickase or nCas) and adenosine deaminase (wild-type tRNA-specific adenosine deaminase, which is TadA from Escherichia coli (wtTadA), and modified TadA (eTadA), which is TadA7.10 evolved to operate on DNA instead of RNA). However, in recent adenine base editors, effects of genome-wide guide RNA (sgRNA)-dependent off-target DNA editing, transcript-wide sgRNA-independent off-target RNA editing, ABE-mediated cytosine deamination at off-target sites, and the like have been reported. The first off-target effect is caused by the incomplete target specificity of Cas nuclease, and the other two off-target effects are attributable to the DNA/RNA-binding properties of adenosine deaminase.

To reduce off-target effects on RNA, several research groups are currently developing new versions of ABE into which additional mutations are introduced. For example, it was reported that the addition of the F148A mutation in both wtTadA and TadA7.10 reduced random RNA deamination activity. Another group independently reported results that wtTadA of ABE7.10 mainly affects the RNA deamination activity. On the other hand, it was found that in the absence of wtTadA, the DNA editing activity was not affected. Thus, it was found that with the introduction of the E59A mutation, wtTadA was inactivated, and an additional mutation (V106W) in TadA7.10 reduced off-target effects on RNA without reducing DNA targeting activity. Another group completely removed wtTadA and added several mutations to TadA7.10 (K20A/R21A or V82G) to reduce RNA off-target effects (Nat Biotechnol 37, 1041-1048 (2019)). However, as described above, studies have been conducted mainly focusing on reducing ABE-mediated RNA deamination activity in many research groups.

On the other hand, cytosine base editors (CBEs) are known to be effective gene editing tools capable of substituting cytosine with thymine without causing DNA double-strand breaks (DSBs) or using a donor DNA template. However, cytosine base editors tend to have a wide operating range (position 3 to 9), so there is a problem in that even undesired base editing occurs. In addition, there is another problem in that the substitution of cytosine with bases other than thymine is inappropriate.

Hence, in the present disclosure, a key mutation capable of enhancing cytosine catalysis in adenosine deaminase (TadA) was found to develop a gene editor capable of enabling efficient and sophisticated cytosine base editing.

### DISCLOSURE

### Technical Problem

The inventors of the present disclosure rationally designed and tested dozens of TadA variants to develop a base editor for further efficient and sophisticated cytosine base editing. As a result, mutations that improved cytosine base editing activity were identified, and based on these results, the present disclosure was completed.

Hence, an objective of the present disclosure is to provide an adenine base editor having increased thymine-cytosine (TC) sequence-specific cytosine base editing activity.

In addition, another objective of the present disclosure is to provide a composition for edition of a thymine-cytosine (TC) sequence-specific cytosine base, the composition containing the adenine base editor and a single guide RNA (sgRNA).

Furthermore, a further objective of the present disclosure is to provide a method of editing a thymine-cytosine (TC) sequence-specific cytosine base, the method including bringing the composition into contact with a target sequence in vitro.

Moreover, yet a further objective of the present disclosure is to provide a kit for editing a thymine-cytosine (TC) sequence-specific cytosine base, the kit including the composition.

However, the technical problem to be achieved by the present disclosure is not limited to the above-mentioned problems, and other problems not mentioned will be clearly understood by those skilled in the art from the following description.

### Technical Solution

In order to accomplish the above objectives of the present disclosure as described above, the present disclosure provides an adenine base editor having increased thymine-cytosine (TC) sequence-specific cytosine base editing activity, the adenine base editor having a form in which an adenosine deaminase variant containing a P48R mutation and CRISPR-associated protein 9 (Cas9) protein are fused.

In one embodiment of the present disclosure, the adenosine deaminase may be TadA7.10.

In another embodiment of the present disclosure, the base editor may be ABEmax into which the mutation is introduced.

In a further embodiment of the present disclosure, the base editor may be further linked with one or more uracil-DNA glycosylases (UGIs).

In addition, the present disclosure provides a composition for edition of a thymine-cytosine (TC) sequence-specific cytosine base, the composition including the adenine base editor and
a single guide RNA (sgRNA).

In one embodiment of the present disclosure, the cytosine may be a cytosine (C) positioned at the 5th, 6th, or 7th base from the 5′ end of a target sequence.

In another embodiment of the present disclosure, in the composition, the cytosine (C) positioned right behind thymine (T) in the target sequence may be substituted with thymine (T) or guanine (G) according to the presence or absence of UGI.

Furthermore, the present disclosure provides a method of editing a thymine-cytosine (TC) sequence-specific cytosine base, the method including bringing the composition into contact with a target sequence in vitro.

Moreover, the present disclosure provides a kit for editing a thymine-cytosine (TC) sequence-specific cytosine base editing, the kit including the composition.

### Advantageous Effects

An adenine base editor having increased TC sequence-specific cytosine base editing activity, according to the present disclosure, was produced by introducing a P48R mutation into an adenosine deaminase. The adenine base editor has an operating range that is more sophisticated than conventional cytosine base editors, allows cytosine base editing only when thymine is positioned right in front of cytosine, thereby enabling elaborate editing even when there is a plurality of cytosines within the range, enables cytosine to be substituted with thymine by adding UGI, and enables cytosine to be substituted with guanine without adding UGI. Therefore, a cytosine base editing composition containing the cytosine base editor and an sgRNA, according to the present disclosure, can be effectively used in the fields of gene therapy or new crop development which requires precise editing of only cytosine in all living organisms, including humans, plants, and bacteria.

### DESCRIPTION OF DRAWINGS

FIG. 1 is a schematic diagram illustrating cytosine base editing and target adenine base editing mediated by the catalytic activity of ABE;

FIG. 2 shows adenosine deaminases contained in a variety of adenine base editors (left) and a graph of the respective base editing efficiencies of ABEs at A4 and C6 in the FANCF and RNF2 sites (right);

FIG. 3 shows analysis results of the amino acid sequences of adenosine deaminases (TadA) and the structures thereof, in which FIG. 3A shows sequence alignment results of wtTadA, TadA7.10, TadA8e, saTadA, and 10 TadA homologous proteins from various species, and FIG. 3B shows the result of a structure in which the apo structure from E. coli (green; PDB code 1Z3A) is superimposed on the holo structure (pink and gray; PDB code 2B3J) of saTadA bound to RNA;

FIG. 4 shows heat map analysis results of adenine and cytosine base editing efficiencies induced by 33 ABE variants in the FANCF and RNF2 sites (left) and a graph of the accuracy values obtained by dividing the adenine base editing efficiency by the cytosine base editing efficiency (right);

FIG. 5 is a diagram schematically illustrating components of conventionally known cytosine base editors (CBE and CBE (ΔUGI)), adenine base editors (ABEmax and ABEmax-UGI), and adenine base editors into which a P48R mutation is introduced (ABE-P48R and ABE-P48R-UGI);

FIG. 6 is a diagram showing heat map results of whether the substitutions of bases on the target sites of the CSRNP3 gene are performed or not, the results obtained by performing high-throughput sequencing after transfecting cells with each of the base editors illustrated in FIG. 5;

FIG. 7 shows analysis results of the tendency of cytosine substitutions in each TC motif of four endogenous genes (FANCF, RNF2, ABLIM3, and CSRNP3), the results obtained by performing high-throughput sequencing after transfecting cells with each of the base editors illustrated in FIG. 5;

FIG. 8 shows examination results of cytosine base editing effects in a TC motif depending on cytosine positions after transfecting cells with adenine base editors (ABE-P48R and ABE-P48R-UGI) into which adenine base editors (ABEmax and ABEmax-UGI) and a P48R mutation are introduced;

FIG. 9 shows examination results of cytosine base editing effects depending on types of motifs after transfecting cells with adenine base editors (ABE-P48R and ABE-P48R-UGI) into which adenine base editors (ABEmax and ABEmax-UGI) and a P48R mutation are introduced;

FIG. 10 is a diagram identifying and schematizing the number of diseases caused by the substitution of thymine with cytosine and diseases caused by the substitution of guanine with cytosine, using the ClinVar database;

FIG. 11 shows comparison results of therapeutic potentials by measuring cytosine base editing efficiency after transfecting cell lines mimicking genetic variations of diseases associated with missense mutations of the TUBB6 gene in which the TT sequence is mutated into the TC with ABE-P48R-UGI and CBE (AncBE4max) of the present disclosure; and

FIG. 12 shows comparison results of therapeutic potentials by measuring cytosine base editing efficiency after transfecting cell lines mimicking genetic variations of non-toxic and toxic goiter diseases associated with missense mutations of the TPO gene in which the TG sequence is mutated into the TC with ABE-P48R and CBE (AncBE4max) of the present disclosure.

### BEST MODE

Hereinafter, the present disclosure will be described in detail.

The inventors of the present disclosure identified a key mutation that significantly reduced adenine base editing activity and increased cytosine base editing activity in an adenine base editor, and introduced the mutation described above into an adenosine deaminase to produce an adenine base editor having increased thymine-cytosine sequence-specific cytosine base editing activity.

The present disclosure provides an adenine base editor having increased thymine-cytosine (TC) sequence-specific cytosine base editing activity, the adenine base editor having a form in which an adenosine deaminase variant containing a P48R mutation and

CRISPR-associated protein 9 (Cas9) protein are fused.

As used herein, the term “base editors (BEs)” refers to tools for editing a single base. More specifically, the base editors are produced by fusing adenosine deaminases or cytosine deaminases to the N-terminus of Cas9 nickase, which are named adenine base editors (ABEs) and cytosine base editors (CBEs), respectively. While the BEs described above do not cause double-strand breaks, ABEs edit adenine to guanine at specific sites, and CBEs edit cytosine to thymine at specific sites.

As used herein, the term “adenine base editors (ABEs)” is produced by fusing any naturally-derived deaminase (ecTadA) and/or adenosine deaminase variants (ecTadA*) to the N-terminus of Cas9 nickase to edit adenine to guanine. Types of ABEs include versions of ABE6.3, ABE7.8, ABE7.9, ABE 7.10, ABEmax, ABEmax-m, SECURE-ABE, ABE8e, ABE8e-V106W, ABE8.17-m, and the like depending on variations or types of adenosine deaminase, but are not limited thereto. The adenine base editor may be referred to as “ABEs”.

In the present disclosure, the adenine base editor may be an improved version having the characteristic of reduced specificity or accuracy for adenine base editing and significantly increased thymine-cytosine sequence-specific cytosine base editing activity. The adenine base editor is preferably an ABEmax version to which the mutation is induced, but is not limited thereto.

As used herein, the term “adenosine deaminase” is an enzyme involved in the removal of an amino group from adenine and the production of hypoxanthine. The enzyme is rarely found in higher animals, but has been reported to be present in small amounts in the muscles of cows, milk, and blood of mice, and in large amounts in the intestines of crayfish, insects, and the like. Adenosine deaminase may include naturally-derived adenosine deaminases, such as ecTadA, or adenosine deaminase variants, such as a mutation of ecTadA (ecTadA*). The variant preferably includes TadA7.10, TadA8e, TadA8s, TadA8.20 or TadA8.17, and the like. In the present disclosure, TadA7.10 was used. However, the variant is not limited thereto.

As used herein, the term “Cas9 (CRISPR-associated protein 9) protein”, a protein that plays a significant role in the immunological defense of certain bacteria against DNA viruses, is being used widely in genetic engineering applications. The protein can be applied to modify the cell genome because the key function thereof is to cleave DNA. Specifically, CRISPR-Cas9 recognizes, cleaves, and edits a specific base sequence to be used as a third-generation gene editor, and is useful when simply, quickly, and efficiently carrying out modifications of inserting a specific gene into a target site in the genome or stopping specific gene activity. The Cas9 protein or gene information thereof may be obtained from a known database, such as GenBank of the National Center for Biotechnology Information (NCBI), but is not limited thereto. In addition, those skilled in the art may appropriately link additional domains with the Cas9 protein depending on the purpose. In the present disclosure, the Cas9 protein may include not only wild-type Cas9 but also Cas9 variants as long as the variants have the function of nuclease for gene editing.

In addition, the origin of the Cas9 protein in the present disclosure is not limited. As non-limiting examples, the Cas9 protein may be derived from Streptococcus pyogenes, Francisella novicida, Streptococcus thermophilus, Legionella pneumophila, Listeria innocua, or Streptococcus mutans. However, the Cas9 protein is preferably from Streptococcus pyogenes.

In the present disclosure, the base editor may be further linked with one or more uracil DNA glycosylases (UGIs) and preferably is further linked with two UGIs. The UGI is also referred to as UNG or UDG and serves to hydrolyze the N-glycosylic bond between a uracil base and a deoxyribose sugar of uracil-containing DNA to create depyrimidine sites. The enzyme is known to hydrolyze single- and double-stranded DNA containing uracil, but not RNA.

Through specific embodiments, the inventors of the present disclosure confirmed that the TC sequence-specific cytosine base editing activity was increased in the improved adenine base editor according to the present disclosure.

More specifically, in one embodiment of the present disclosure, whether ABE variants, further improved versions compared to ABE7.10, exhibited cytosine deamination activity was examined based on the results confirmed in earlier studies. As a result, it was confirmed that cytosine base editing, in addition to adenine base editing activity, was still induced in all of the tested ABE variants (see Example 2).

In another embodiment of the present disclosure, to examine a mutation capable of affecting the removal of cytosine base editing activity through the distinction between adenine and cytosine in adenosine deaminase, variants in which various candidate mutations were each independently introduced into TadA7.10 of ABEmax or ABEmax-m based on the analysis results of the tertiary structures and sequences of the TadA homologous proteins. Then, adenine and cytosine base editing efficiencies at the target site in HEK293T cells were analyzed. As a result, contrary to expectations, it was found that the P48R mutation in TadA7.10 of ABEmax-m substantially reduced the adenine base editing efficiency, while increasing the cytosine base editing efficiency, resulting in high specificity for cytosine base editing (see Example 3).

In a further embodiment of the present disclosure, cells were transfected with a total of 6 versions including conventionally known cytosine base editors (CBE and CBE (ΔUGI)), adenine base editors (ABEmax and ABEmax-UGI), and improved adenine base editors (ABE-P48R, ABE-P48R-UGI) of the present disclosure produced by introducing a P48R mutation to analyze bases substituted in the target sequence of the endogenous CSRNP3 gene. As a result, in the case of the improved version of the adenine base editors (ABE-P48R and ABE-P48R-UGI) according to the present disclosure, it was confirmed that cytosine positioned at the 6th base from the 5′ end while being mainly positioned right behind thymine, not adenine, was substituted with high efficiency (see Example 4).

In yet a further embodiment of the present disclosure, to verify the therapeutic potentials of ABE-P48R and ABE-P48R-UGI for genetic diseases, a cell line mimicking missense mutations of the TUBB6 gene in which the TT sequence is mutated into the TC and a cell line mimicking missense mutations of the TPO gene in which the TG sequence is mutated into the TC were transfected with ABE-P48R-UGI and CBE (AncBE4max), or ABE-P48R and CBE (AncBE4max) of the present disclosure. Then, cytosine base editing efficiency was measured. As a result, the respective cytosine base editing functions of ABE-P48R-UGI and ABE-P48R that were sophisticated compared to that of CBE were confirmed. Through this result, the therapeutic potential for genetic diseases was seen (see Example 5).

Therefore, the results described above verify the significantly sophisticated cytosine base editing effect of the adenine base editor and the use thereof according to the present disclosure.

Hence, as another aspect of the present disclosure, the present disclosure provides a composition for edition of a thymine-cytosine (TC) sequence-specific cytosine base, the composition including the adenine base editor and
a single guide RNA (sgRNA).

In the cytosine base editing composition of the present disclosure, the cytosine (C) positioned right behind thymine (T) in the target sequence may be substituted with thymine (T) or guanine (G) according to the presence or absence of UGI. More specifically, in the presence of UGI, the cytosine positioned right behind the thymine may be substituted with thymine. In addition, in the absence of UGI, the cytosine positioned right behind the thymine may be substituted with guanine.

In the present disclosure, the cytosine may be a cytosine (C) positioned at the 5th, 6th, or 7th base from the 5′ end of the target sequence. The cytosine is preferably a cytosine positioned at the 5th or 6th base and more preferably, a cytosine positioned at the 6th base, but is not limited thereto.

The “guide RNA (gRNA)” is a single-stranded RNA that serves to find the position of a specific target DNA to be edited and guide the Cas protein to the target DNA. The guide RNA is adjacent to a protospacer adjacent motif (PAM) site and may include a sequence complementary to the 10 to 25-bp base sequence of the DNA to be edited.

The present disclosure provides a method of editing a thymine-cytosine (TC) sequence-specific cytosine base, the method including bringing the composition into contact with the target sequence in vitro.

In the base editing method according to the present disclosure, the target sequence may include a target base to be edited. In addition, the target base to be edited may be a base other than cytosine associated with illnesses or diseases, and is preferably a base in which guanine is point mutated into cytosine, but is not limited thereto.

Furthermore, the present disclosure provides a kit for editing a thymine-cytosine (TC) sequence-specific cytosine base, the kit including the above composition.

In the present disclosure, the kit includes all materials (reagents) necessary for performing base editing, such as a buffer and deoxyribonucleotide-5-triphosphate (dNTP), in addition to the base editing composition. In addition, the optimum amount of reagents used in a particular reaction of the kit can be easily determined by those skilled in the art acquired the present disclosure.

### MODE FOR INVENTION

Hereinafter, preferred embodiments will be presented to aid understanding of the present disclosure. However, the following embodiments are provided to more easily understand the present disclosure, and the content of the present disclosure is not limited to the following embodiments.

### EXAMPLE

### Example 1. Experiment Materials and Experiment Method

1-1. Construction of Plasmids for Expression of ABE Variants

The inventors of the present disclosure constructed plasmids for the expression of ABE variants based on pCMV_ABEmax_P2A_GFP (Addgene #112101), pCMV-ABEmax (TadA E59A) (Addgene #125648), pCMV-ABEmax (TadA, eTadAE59A) (Addgene #125662), pCMV-ABEmax (TadA E59A, eTadAR47Q) (Addgene #125657), pCMV-ABEmax (TadA E59A, eTadAD108Q) (Addgene #125655), pCMV-ABEmaxAW (Addgene #125647), ABE8e (Addgene #138489), ABE8e (TadA-8e V106W) (Addgene #138495), or ABE8.17-m (Addgene #136298). To obtain a vector for ABE expression, 1 μg of pCMV_ABEmax_P2A_GFP 1 μg, 1 unit of NotI-HF (New England Biolabs, catalog number: R3189L), and 1 unit of BglII (Enzynomics, catalog number: R010S) were mixed in a final volume of 50 μL. Subsequently, after incubating the mixture at a temperature of 37° C. for 1 hour to induce a cleavage reaction, electrophoresis was performed to separate the cleaved product according to sizes, and a gel extraction kit, Expin™ Gel SV mini (GeneAll, catalog number: 102-102), was used for purification. 2 U of T5 exonuclease (New England Biolabs, catalog number: M0363S), 12.5 U of Phusion DNA polymerase (Thermo Fisher Scientific, catalog number: F530L), 2 kU of Taq DNA ligase (New England Biolabs, catalog number: M0208S), 0.2 M Tris-HCl (pH 7.5), 0.2 M MgCl₂, 2 mM dNTPs, 0.2 M dithiothreitol, 25% PEG-8000, and 1 mM NAD were mixed in a final volume of 10 μL. Then, the purified product and the synthesized PCR product containing variants to be identified were added to the mixture, mixed, and incubated at a temperature of 50° C. for 1 hour. Next, the resulting product was transformed into 100 μL of DH5a competent cells. A single transformed colony was inoculated into an LB medium containing antibiotics, and the plasmid was isolated from the cells using a DNA prep kit (Enzynomics, EP101-200N).

1-2. Production of ABE-UGI Through DNA Cloning Using CRISPR-Cas9

DNA cloning using CRISPR-Cas9, without restriction enzymes, was performed by partially modifying conventionally known methods. More specifically, an sgRNA was designed to cleave a sequence encoding the C-terminus of Cas9 (D10A) in pCMV_ABEmax_P2A_GFP. In addition, for in vitro DNA cleavage, 0.7 μg of an in vitro transcribed sgRNA and 1 μg of recombinant NG-SpCas9 were pre-incubated at room temperature for 5 minutes. Then, 1 μg of pCMV_ABEmax_P2A_GFP and water treated with DEPC was added to the SpCas9-sgRNA and set to have the final volume of 50 μL. Next, the mixture was incubated at a temperature of 37° C. for 30 minutes to induce cleavage, and the product was then purified by electrophoresis. The inserted PCR product was amplified from pCMV_AncBE4max (Addgene plasmid #112094). The two fragments were linked by Gibson assembly.

1-3. Transfection Using Liposomes

HEK293T (ATCC®CRL-3216™) cells were incubated in a DMEM medium supplemented with 10% FBS and 1% ampicillin under conditions: a temperature of 37° C. and an atmosphere of 5% CO₂. Cell density was estimated through a hemocytometer and microscopic observation. Before transfection, the HEK293T cells were dispensed in a 24-well plate at a density of 1×10⁵ cells per well, incubated for 24 hours, and treated with a mixed solution of 2 μL of lipofectamine® 2000 reagent (Thermo Fisher Scientific, 11668019), 1 μL of plasmid DNA (750 ng of an ABE expression plasmid and 250 ng of an sgRNA expression plasmid), and a serum-free medium. Next, genomic DNA was isolated 72 hours after transfection.

1-4. Transfection by Electroporation

Using Neon™ Transfection System 10 μL kit (Thermo Fisher Scientific, MPK1025), an ABEmax expression plasmid (500 ng) and an sgRNA expression plasmid (170 ng) were introduced into 2×10⁵ cells by electroporation. Appropriate electroporation parameters (1,500 V-20 ms-2 pulses for HEK293T cells) were compliant according to the manufacturer's protocol for progress. In addition, genomic DNA was isolated 72 hours after transfection.

1-5. Sequence Alignment of TadA Homologous Proteins

TadA homologous proteins (orthologs) were found using protein BLAST in the National Center for Biotechnology Information (NCBI). E. coli wtTadA sequence (GenBank ID: WP_001297409.1) was used as an input sequence, and 10 homologous proteins exhibiting up to 40% of sequence identity were selected. The accession numbers and species for the selected sequences are as follows: vsTadA, WP_127165941.1, Veillonella sp. CHU732; ssTadA, WP_105128341.1, Streptococcus suis; asTadA, WP_067866801.1, Acinetobacter sp. SFB; bfTadA, WP_073388705.1, Butyrivibrio fibrisolvens; sxTadA, WP_107541930.1, Staphylococcus xylosus; cbTadA, WP_111988333.1, Cellvibrionaceae bacterium AOL6; dfTadA, WP_117494436.1, Dorea formicigenerans; ocTadA, WP_047980683.1, Ornithinibacillus contaminans; osTadA, WP_077602817.1, Oceanobacillus sojae; and pgTadA, WP_026908502.1, Paucisalibacillus globulus.

1-6. Targeted Deep Sequencing

HEK293T cells were centrifuged. Next, the cell pellet was resuspended in 100 μL of proteinase K extraction buffer solution [40 mM Tris-HCl (pH 8.0), 1% Tween-20, 0.2 μM EDTA, mg of Proteinase K, and 0.2% Nonidet P-40], incubated at a temperature of 60° C. for 15 minutes, and then incubated at a temperature of 98° C. for 5 minutes. Genomic DNA isolated from the HEK293T cells was amplified using KOD-Multi & Epi (TOYOBO, KME-101). The resulting PCR product was analyzed using an Illumina Mini-Seq instrument. The results of Mini-seq were analyzed using BE-Analyzer (http://www.rgenome.net/be-analyzer/).

1-7. Targeted RNA Sequencing

In a 24-well plate, HEK293T cells were transfected with 500 ng of an ABE expression plasmid and 170 ng of an sgRNA expression plasmid by electroporation. After 24 hours, the cells were washed with DPBS. RNA was isolated using a NucleoSpin® RNA Plus kit (MACHEREY-NAGEL, 740984. 250) according to the manufacturer's protocol. In addition, cDNA synthesis through reverse transcription was performed using ReverTra Ace-α-™ (TOYOBO, FSK-101) according to the manufacturer's instructions. For cDNA amplification, PCR was performed using KOD-Multi & Epi (TOYOBO, KME-101), and an Illumina Mini-Seq instrument was used to analyze a PCR product. To obtain the editing ratio of adenosine to inosine, the number of adenosine to guanosine conversions in the product was divided by the total number of adenosines.

### Example 2. Measurement of Cytosine Deamination Activity Against Various ABE Variants

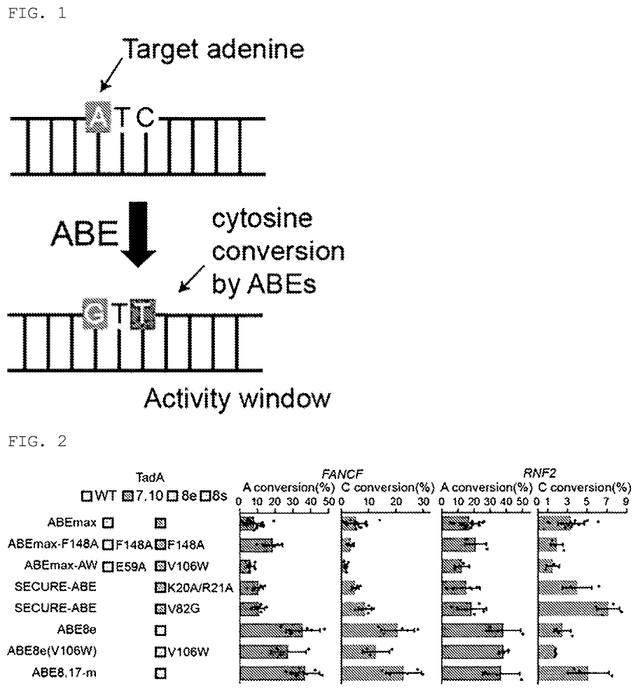

From earlier studies, the inventors of the present disclosure confirmed that ABE7.10, an adenine base editor (ABE), was able to substitute cytosine by catalyzing cytosine deamination in the presence of a TC target motif, in addition to editing adenine, the original target base, within a narrow operating range (positioned at the 5th, 6th, or 7th base from the 5′ end of a Cas9 targeting range) of the preferred motif (TC*N), as illustrated in FIG. 1. In addition, it was confirmed that cytosine base substitution by such cytosine deaminase appeared not only in ABE7.10, but also in its earlier versions (ABE6.3, ABE7.8, and ABE7.9) and further optimized versions (ABEmax).

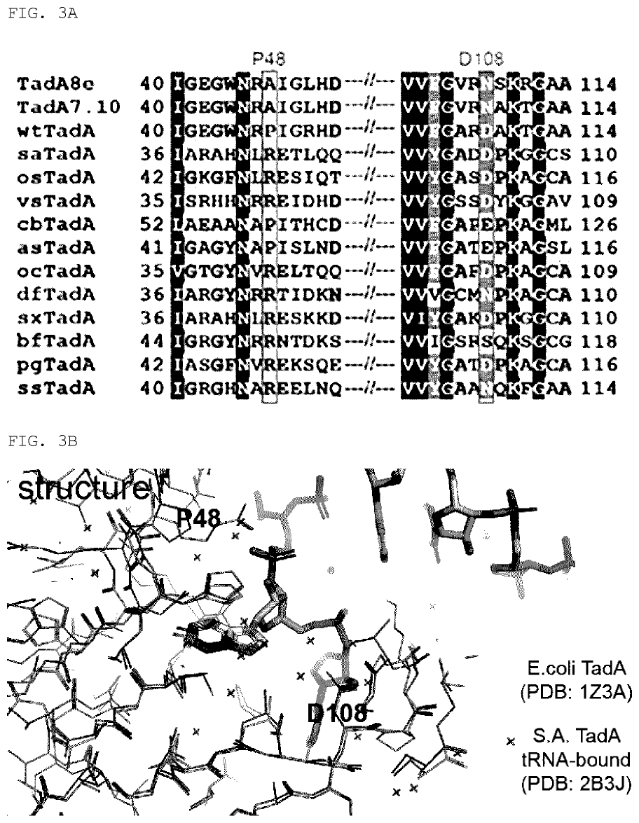

Hence, the inventors of the present disclosure have recently attempted to examine whether cytosine deamination activity is also exhibited in ABE variants developed through various improvements of TadA, adenosine deaminase, for various purposes. The types of ABE variants used in this experiment are as follows, and specific TadA information included in each variant is shown in FIG. 2: 1) versions developed for the purpose of reducing the ABE-mediated RNA off-target effect (for example, ABEmax-F148A, ABEmax-AW, and SECURE-ABEs), 2) versions containing TadA8e variants exhibiting increased deamination activity (for example, ABE8e and ABE8e-V106W), and 3) versions containing TadA8s exhibiting enhanced editing activity (for example, ABE8.17-m).

To this end, all ABE variants were tested on two representative endogenous targets (FANCF and RNF2) containing both cytosine target motifs and adenine residues within the editing operating range in human HEK293T cells. High-throughput sequencing was performed to obtain the following results.

First, as seen in FIG. 2, all the ABE variants tested were still exhibited to induce cytosine base editing in addition to adenine base editing. However, in the case of ABEmax-F148A and ABEmax-AW, the cytosine base editing efficiency was confirmed to be slightly reduced. Such results were determined to support the hypothesis of the inventors of the present disclosure that additional engineering of eTadA enabled the removal or minimization of cytosine deamination activity.

Second, all the ABE8 variants exhibited not only greatly increased adenine base editing efficiency compared to earlier versions but also increased cytosine base editing efficiency. Nevertheless, in the case of ABE8e-V106W, it was confirmed that the cytosine base editing efficiency was reduced compared to ABE8e.

Third, it was seen that the inactivation or removal of wtTadA did not interfere with the DNA editing activity. Rather, the ABEmax version free of wtTadA (ABEmax-m) exhibited higher DNA editing activity than the original ABEmax.

### Example 3. Identification of Mutations in TadA that Significantly Affect Cytosine Base Editing Efficiency

The inventors of the present disclosure attempted to identify a key mutation capable of promoting the distinction between adenine and cytosine in TadA, adenosine deaminase, and considered that some of TadA homologous proteins were already able to be evolved to avoid cytosine base editing. Therefore, to this end, the amino acid sequences of TadA homologous proteins derived from various species were examined.

As a result, as shown in FIG. 3A, based on the aligned amino acid sequence of each TadA homologous protein and the tertiary structure of Staphylococcus aureus TadA (saTadA) bound to a tRNA fragment in FIG. 3B, it was found that various residues inside and outside the active sites were substantially mutated in between the homologous proteins. For example, it was seen that E. coli wtTadA was substituted with arginine in most of the TadA homologous proteins, and D108 was substituted with asparagine, glutamate, or serine in other homologous proteins. In addition, the saTadA structure in FIG. 3B provided insight into the structural changes of an RNA substrate required for the deamination of cytosine smaller than adenine. For adenine deamination activity, the hexagonal ring of adenine needs to be positioned deep inside an adenine-binding pocket, similar to what is seen in the saTadA structure in which a purine base is bound to a pocket. However, cytosine deamination requires a pyrimidine ring in the structure to be in the same position as the hexagonal ring of the purine base, so the sugar-phosphate backbone needs to shift to the edge of the pocket. Therefore, the inventors of the present disclosure determined to substitute P48 and D108 with larger residues capable of preventing the DNA backbone from accessing the rim of the pocket. In addition, V30 and F84 positioned in the adenine-binding pocket were substituted with isoleucine and leucine found at the corresponding positions in various TadA homologous proteins. Furthermore, mutations that were tested earlier and exhibited to incompletely reduce RNA editing activity, such as R47Q mutation maintaining DNA on-target editing activity and D53E mutation reducing RNA editing activity in vitro, were introduced together.

Next, each of the above candidate mutations capable of affecting the cytosine base editing effect of ABE was introduced into TadA7.10 of ABEmax or ABEmax-m, and then transfected into HEK293T cells to test the nucleotide conversion activity of each ABE variant at a target site in the FANCF and RNF2 genes.

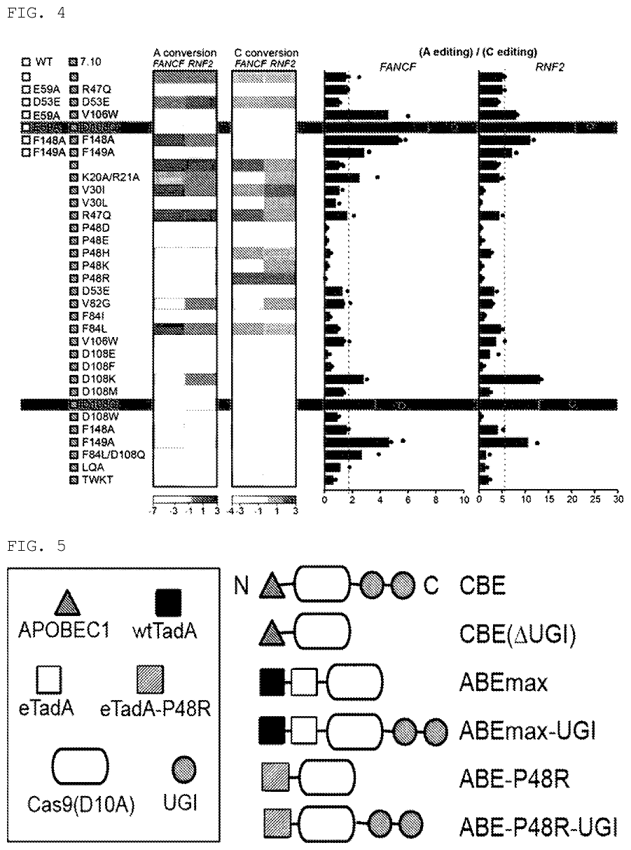

As a result, as shown in FIG. 4, when the adenine and cytosine base editing efficiencies of the ABE variants were normalized to the efficiency of ABEmax, it was confirmed that the adenine and cytosine base editing efficiencies increased or decreased simultaneously in most of the variants. However, the inventors of the present disclosure unexpectedly found out that in TadA7.10 of ABEmax-m, the P48R mutation substantially reduced the adenine base editing efficiency while increasing the cytosine base editing efficiency, resulting in high specificity for cytosine base editing.

Through the above results, TadA7.10-P48R, a TadA7.10 variant exhibiting improved selectivity for cytosine base editing, was found.

### Example 4. Confirmation of TC-Specific Cytosine Base Editing Effect of Adenine Base Editor into which P48R Mutation is Introduced

Based on the results of Example 3, the inventors of the present disclosure attempted to develop a TC sequence-specific base editing tool capable of significantly reducing adenine base editing activity while increasing cytosine base editing activity, using a TadA7.10-P48R variant into which a P48R mutation was introduced. To this end, two copies of uracil DNA glycosylase (UGI) were linked to the C-terminus of ABEmax-P48R, as in AncBE4max, an optimized cytosine base editor (CBE). Adding UGI may increase the efficiency of editing cytosine (C) to thymine (T), not the efficiency of editing the cytosine (C) to guanine (G), or vice versa. Hence, the inventors of the present disclosure ultimately prepared the following six types of TC-specific base editors. Components thereof are schematically illustrated in FIG. 5: AncBE4max and AncBE4max (ΔUGI), which are types of CBEs; ABEmax and ABEmax-UGI, which are types of ABEs; and ABEmax-P48R and ABEmax-P48R-UGI, which are types of ABEs into which P48R is introduced.

After transfecting HEK293T cells with the above 6 types of TC-specific base editors, a base editing test was conducted at a target site of the CSRNP3 gene, and high-throughput sequencing was then performed.

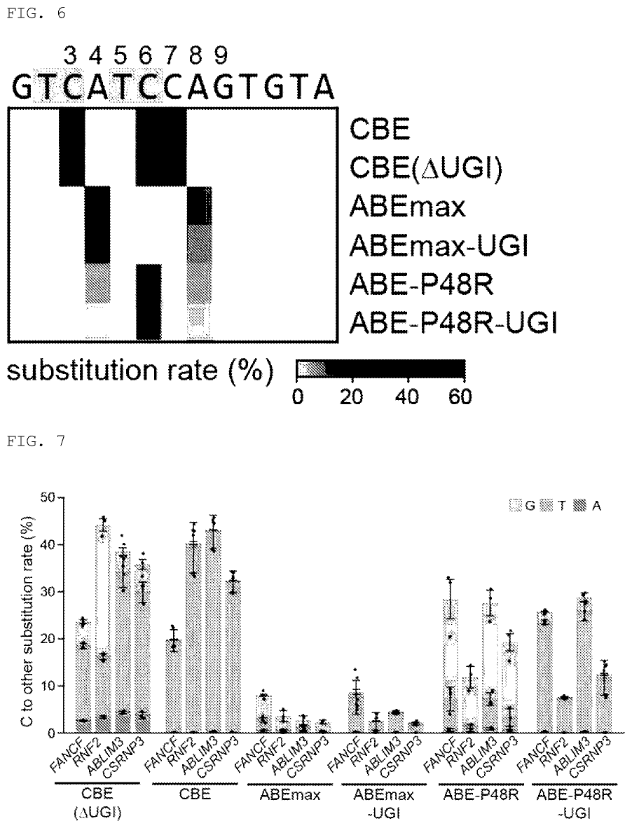

As a result, as seen from the heat map results in FIG. 6, CBE and CBE (ΔUGI) substituted all types of cytosine (C) (for example, C3, C6, and C7) at the highest rate within the editing operating range. In addition, ABEmax and ABEmax-UGI exhibited to substitute all types of A (for example, A4 and A8) and C6. On the other hand, ABE-P48R and ABE-P48R-UGI were confirmed to mainly substitute C6.

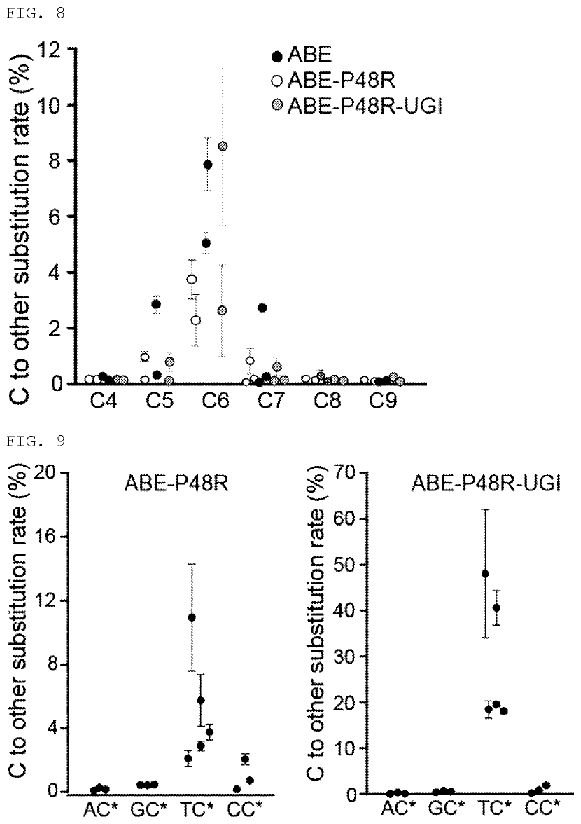

Therefore, the inventors of the present disclosure conducted a test by adding target sites of three endogenous genes (FANCF, RNF2, and ABLIM3) in addition to the CSRNP3 gene, and then performed high-throughput sequencing. As a result, as shown in FIG. 7, the tendency of editing activity coincided with the results at the target site of the CSRNP3 gene. While in the case of ABE-P48R, the substitution of C with G was mainly seen, in the case of ABE-P48R-UGI, the substitution of C with T was seen, as expected. As shown in FIG. 8, when treating a target having a TC motif at a different position, such cytosine substitution effect was able to be applied to cytosine positioned at the 5th, 6th, or 7th base from the 5′ end. In addition, as shown in FIG. 9, it was confirmed that the cytosine base editing effect occurs specifically to the TC motif-containing target.

These results imply that ABE-P48R and ABE-P48R-UGI can each independently function as editing tools for the substitution of cytosine adjacent directly to thymine with guanine and thymine (TC-to-TG and TC-to-TT).

### Example 5. Verification of Therapeutic Potentials for Genetic Diseases Using Adenine Base Editors Having Increased TC-Specific Cytosine Base Editing Activity

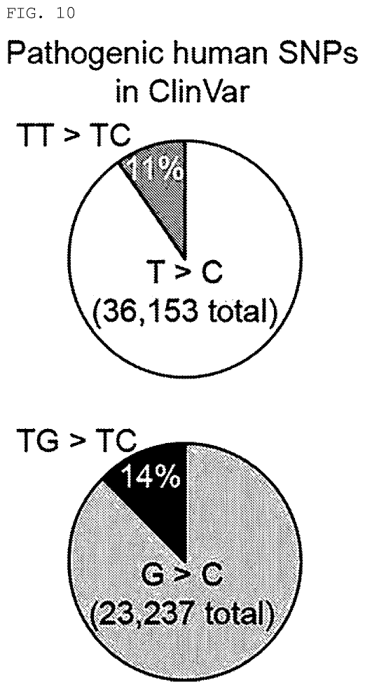

To verify the therapeutic potentials of ABE-P48R and ABE-P48R-UGI for genetic diseases, the inventors of the present disclosure examined all the targeted mutations registered in the in silico ClinVar database. As seen in FIG. 10, a total of 36,153 T>C mutations that caused pathological phenotypes in the database were positioned within the standard cytosine base editing operating range (4th to 8th positions). Among these, 3,874 (11%) mutations were associated with cytosine targeting motifs within the editing operating range of ABE-P48R-UGI. In addition, it was confirmed that 3,248 (14%) of 23,237 G>C mutations in the database were able to be targeted by ABE-P48R.

5-1. TC-to-TT Editing Using ABE-P48R-UGI

For example, missense mutations in the TUBB6 gene (causing an F394S change in the protein) are associated with congenital facial paralysis, bilateral ptosis, and velopharyngeal dysfunction. For radical treatment, the TC sequence needs to be edited to TT. Hence, the inventors of the present disclosure first established a cell line containing appropriate mutations in the genome to mimic the genetic variations of the diseases. Next, CBE (AncBE4max) or ABE-P48R-UGI was each independently transfected into the cell line, and high-throughput sequencing was then performed.

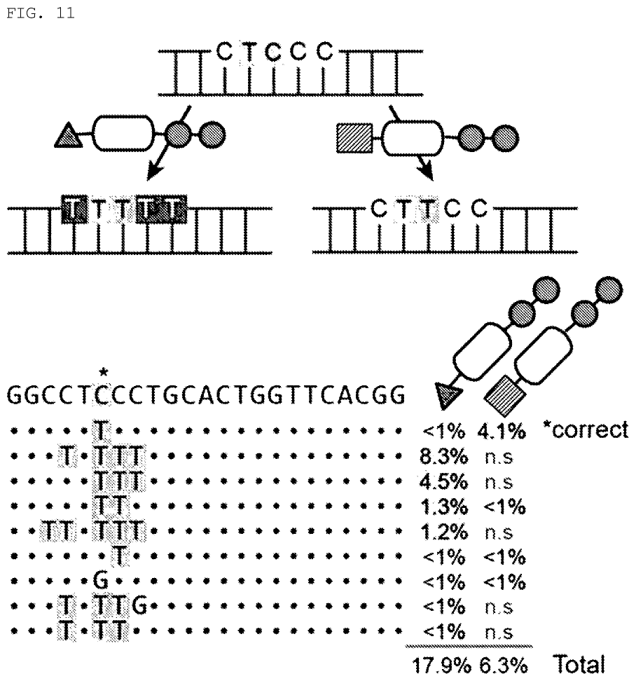

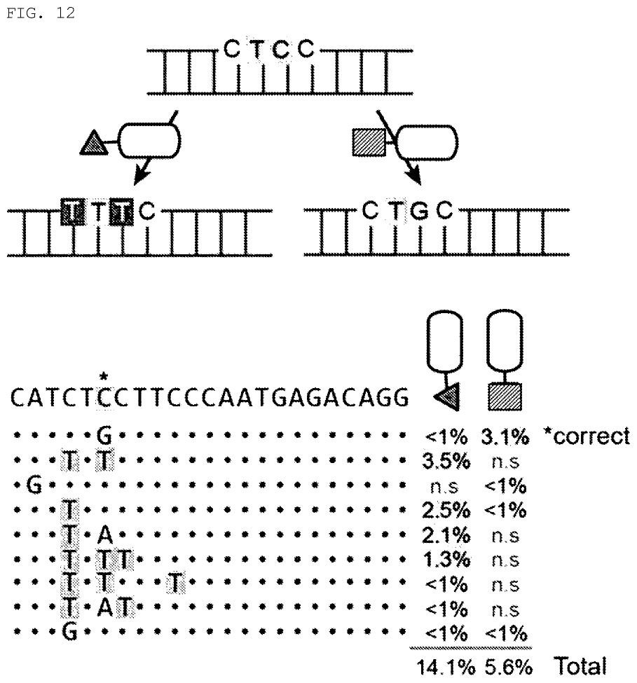

As a result, as shown in FIG. 11, it was confirmed that CBE exhibited higher total cytosine base editing efficiency than ABE-P48R-UGI but lower precise editing efficiency (ABE-P48R-UGI exhibited 4.1%, and CBE exhibited less than 1%). In addition, it was confirmed that ABE-P48R-UGI exhibited a negligible bystander effect while CBE exhibited a significant bystander effect. Therefore, the above results imply that ABE-P48R-UGI can be used as an effective editing tool for TC-to-TT editing.

5-2. TC-to-TG Editing Using ABE-P48R

In the case of missense mutations in the TPO gene (causing a Q660E change) found in both nontoxic and toxic goiter patients, the TC sequence needs to be edited to TG for treatment. Hence, the inventors of the present disclosure established a cell line containing appropriate mutations in the genome as described above to mimic the genetic variations of the diseases. Next, CBE (AncBE4max) or ABE-P48R was each independently transfected into the cell line, and high-throughput sequencing was then performed.

As a result, as shown in FIG. 12, it was confirmed that CBE exhibited higher total cytosine base editing efficiency than ABE-P48R but lower precise editing efficiency (ABE-P48R exhibited 3.1%, and CBE exhibited less than 1%). In addition, while ABE-P48R exhibited a negligible bystander effect like ABE-P48R-UGI, CBE exhibited a significant bystander effect. Therefore, the above results imply that ABE-P48R can be used as an effective editing tool for TC-to-TG editing.

1. An adenine base editor having increased thymine-cytosine (TC) sequence-specific cytosine base editing activity, the adenine base editor having a form in which an adenosine deaminase variant comprising a P48R mutation and CRISPR-associated protein 9 (Cas9) protein are fused.

2. The base editor of claim 1, wherein the adenosine deaminase is TadA7.10.

3. The base editor of claim 1, wherein the base editor is ABEmax into which the mutation is introduced.

4. The base editor of claim 1, wherein the base editor is further linked with at least one uracil-DNA glycosylase (UGI).

5. A composition for edition of a thymine-cytosine (TC) sequence-specific cytosine base, the composition comprising:
the adenine base editor of claim 1; and
a single guide RNA (sgRNA).

6. The composition of claim 5, wherein the cytosine is a cytosine (C) positioned at the 5th, 6th, or 7th base from the 5′ end of a target sequence.

7. The composition of claim 5, wherein the cytosine (C) positioned right behind thymine (T) in a target sequence is substituted with thymine (T) or guanine (G) according to the presence or absence of UGI.

8. A method of editing a thymine-cytosine (TC) sequence-specific cytosine base, the method comprising bringing the composition of claim 5 into contact with a target sequence in vitro.

9. A kit for editing a thymine-cytosine (TC) sequence-specific cytosine base, the kit comprising the composition of claim 5.
